(19) United States
(12) Patent Application Publication
(10) Pub. No.: US 2007/0016083 A1
(43) Pub. Date: Jan. 18, 2007

(54) Arterial stiffness evaluation apparatus, and arterial stiffness index calculating program

(76) Inventor: Motoharu Hasegawa, Tokyo (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 11/482,707

(22) Filed: Jul. 10, 2006

(30) Foreign Application Priority Data
Jul. 11, 2005 (JP) 2005-201683

(51) Int. Cl. A61B 5/02; A61B 5/103

(52) U.S. Cl. 600/483; 600/587

(57) ABSTRACT

Provided is an arterial stiffness evaluation apparatus using a new arterial stiffness index which serves as an index to evaluate a degree of arterial stiffness, and a program therefor. The arterial stiffness apparatus includes pulse wave detecting means, pulse wave propagation velocity deciding means, blood pressure detecting means, and arterial stiffness index calculating means, in which the arterial stiffness index calculating means executes: a first step of calculating a pulse wave propagation velocity PWVori after pressure calibration and CAVI=ln(Ps/Pd)×PWV², and deriving a regression equation where PWVori is represented by a quadratic equation of CAVI; a second step of setting the regression equation as an equation representing a arterial stiffness index PWVpcm1; a third step of deriving a regression equation where the PWVori is represented by a linear equation of the PWVpcm1; a fourth step of setting the regression equation as an equation representing an arterial stiffness index PWVpcm2; and a fifth step of obtaining a PWVpcm2 value by substituting a measured value, and the degree of arterial stiffness is evaluated based on the PWVpcm2.

### BACKGROUND OF THE INVENTION

1. Field of the Invention

The present invention relates to an arterial stiffness evaluation apparatus for evaluating a degree of angiosclerosis, especially, arterial stiffness of an organism, and to an arterial stiffness index calculation program used for the apparatus.

2. Description of the Related Art

Several methods for diagnosing arterial stiffness have been developed. Among such methods, there is a method of using a pulse wave propagation velocity (or pulse wave velocity which is also referred to as “PWV”). A pulse wave refers to pulse motion generated when a change in pressure in a blood vessel due to extrusion of blood from a heart to a main artery through a contraction of the heart propagates in a peripheral direction, and the pulse wave propagation velocity refers to a propagation velocity of the pulse wave through a blood vessel. A healthy blood vessel is soft and elastic, while an arteriosclerotic blood vessel is stiff and brittle. According to the method, the propagation velocity of the pulse wave in an artery is measured to diagnose the blood vessel as being in a greater progress of angiosclerosis as the propagation velocity is faster, by using the nature of the pulse wave which propagates fast through a stiff substance but slow through a soft substance.

As a representative method of using the pulse wave propagation velocity, there has been known a main artery pulse wave velocity method (hereinafter, referred to as “PWV original method”). According to the PWV original method, a pulse wave propagation velocity is measured for a portion from a main artery valve port to a common iliac artery inguinal part. With respect to a test subject who is laid on the back, a II heart sound, a carotid pulse wave, and a tinea pulse wave are simultaneously recorded along a time axis, and a blood pressure is measured based on Korotkoff sound in a brachial artery. Pulse wave propagation time T from the main artery valve port to the common iliac artery inguinal part is represented by T=tc+tcf, where tc is a time difference from the generation of the heart II sound to when a carotid pulse wave descending leg cut is observed, and tcf is a time difference from a rising point of the carotid pulse wave to a rising time of the tinea pulse wave. On the other hand, it is known that an artery length L from the main artery valve port to the common iliac artery inguinal part is obtained by multiplying I by 1.3, where I is a direct distance between a II intercostals substernal edge and an opposite side tinea pulse beat part, in other words, L=I×1.3 holds true. As described above, a pulse wave propagation velocity PWV′ from the main artery valve port to the common iliac artery inguinal part (hereinafter, pulse wave velocity not pressure-calibrated will be referred to as “PWV”) is obtained by L/T, in other words, PWV′=1.3×l/tc+tcf. As the pulse wave propagation velocity PWV′ thus obtained takes a value that changes according to a blood pressure, a pulse wave propagation velocity PWVori after pressure calibration is obtained as an index where calibration is carried out based on a blood pressure to remove pressure effects. This is how the PSV original method works.

The pulse wave propagation velocity PWVori pressure-calibrated by the PWV original method has been regarded as an arterial stiffness index unique to an individual, which makes it possible to compare many test subjects simultaneously, and to evaluate a change in one test subject over a long time. In particular, the main artery targeted by the PWV original method is a central elastic type, which precedes arteriolopathy changes of other organs in terms of systematic arterial stiffness distribution characteristics, so its predictability is valued.

There has also been known a method of using a neck artery system phase tracing type ultrasonic wave displacement method (hereinafter, referred to as “β method”) as an index which is not based on a pulse wave propagation velocity to evaluate arterial stiffness. An equation ln(Ps/Pd)=β(Ds−Dd)/Dd has experimentally been proven to hold true, where Ds is an outer diameter of a blood vessel in the case of a highest blood pressure Ps and Dd is an outer diameter of a blood vessel in the case of a lowest blood pressure Pd. β derived from the equation is called a stiffness parameter, and β=ln(Ps/Pd)×D/ΔD is used as an index to represent a degree of arterial stiffness. With the β method adopted to actual measurement of a test subject, the subject is laid on the back to whom an ultrasonic wave is applied to the neck part to reproduce an image from a reflected echo, and a phase tracing system is operated to record an aperture micro displacement waveform of a blood vessel. An artery targeted in this measurement is a neck part artery system such as a common carotid artery, a carotid sinus, an internal carotid artery, or a vertebral artery.

In the case of the β method, after its practical application, noninvasive diagnosis of a degree of arterial stiffness of the neck artery has covered a common carotid artery, a sinus artery, an internal carotid artery, and a vertebral artery, and its clinical effects have been highly valued.

The PWV original method and the β method that are indexes for evaluating arterial stiffness both have drawbacks. That is, as the value of the pulse wave propagation velocity fluctuates depending on a blood pressure value, calibration based on the blood pressure value must be carried out in the case of the PWV original method, and a super-micro blood vessel aperture variation (ΔD) must be detected in the case of the β method. In both cases, there has been such a problem that the detector is special and highly expensive.

To solve the drawbacks, the inventors have proposed, in JP 2004-236730 A, a CAVI which is represented by the following equation (1) as an index for evaluating arterial stiffness.
CAVI=k·ln(Ps/Pd)·PWV² (1)
(where Ps indicates a highest blood pressure, Pd indicates a lowest blood pressure, PWV′ indicates a pressure-noncalibrated pulse wave propagation velocity, and k indicates a constant).

A relation is established in a predetermined blood vessel where a square of the pulse wave propagation velocity (PWV′²) increases symmetrically with a decrease of logarithmic pulse pressure (ln(Ps/Pd)). Accordingly, a product of the logarithmic pulse pressure and the square of the pulse wave propagation velocity takes a value unique to the blood vessel. Thus, the use of the CAVI obtained by the equation (1) as the arterial stiffness index leads to advantages that a blood pressure value obtained by eliminating factors caused by blood pressure value fluctuation can be directly used for evaluation and that a result of the evaluation is hardly affected by characteristics of an individual test subject or conditions during measurement. Therefore, the CAVI is an accurate, universal, and objective arterial stiffness index. It should be noted that the logarithmic pulse pressure (ln(Ps/Pd)) is a logarithm of a ratio of a highest blood pressure (systolic blood pressure: Ps) to a lowest blood pressure (diastolic pressure: Pd). However, in the case of the CAVI, a CAVI value shows greater change in a high CAVI value range in which arterial stiffness is suspected, as compared with the arterial stiffness index of the PWV original method, and there is a fear that a doctor or a nurse accustomed to the index of the PWV original method may diagnose a degree of arterial stiffness as being higher than the actual degree from a result of the CAVI.

### SUMMARY OF THE INVENTION

The present invention therefore has an object to provide an arterial stiffness evaluation apparatus which uses a newer arterial stiffness index in place of the CAVI, and an arterial stiffness index calculation program used for the apparatus.

That is, the present invention provides an arterial stiffness apparatus, including: pulse wave detecting means; pulse wave propagation velocity deciding means; blood pressure detecting means; and arterial stiffness index calculating means for calculating an arterial stiffness index to evaluate a degree of arterial stiffness based on a pulse wave propagation velocity and a blood pressure, in which the arterial stiffness index calculating means calculates a PWVpcm2 value by executing: a first step of calculating a pulse wave propagation velocity PWVori after pressure calibration and CAVI=ln(Ps/Pd)×PWV² based on highest blood pressure values Ps, lowest pressure values Pd, and pulse wave propagation velocities PWV′ before pressure calibration obtained from many test subjects, and deriving a regression equation where PWVori is represented by a quadratic equation of CAVI based on a relation between many PWVori values thus obtained and a CAVI value; a second step of setting the regression equation obtained in the first step as an equation representing a arterial stiffness index PWVpcm1; a third step of obtaining a PWVpcm1 value by substituting the CAVI value calculated in the first step into the equation representing the PWVpcm1 obtained in the second step, and deriving a regression equation where the PWVori is represented by a linear equation of the PWVpcm1 based on a relation between the PWVori value and the PWVpcm1 value; a fourth step of setting the regression equation obtained in the third step as an equation representing an arterial stiffness index PWVpcm2; and a fifth step of substituting the highest blood pressure value Ps, the lowest blood pressure value Pd, and the pulse wave propagation velocity PWV′ obtained by the pulse wave detecting means, the pulse wave propagation velocity deciding means, and the blood pressure detecting means, into the equation representing the PWVpcm2 obtained in the fourth step; and the degree of arterial stiffness is evaluated based on the PWVpcm2. Further, the present invention provides an arterial stiffness index calculation program for causing a computer to execute: a process of calculating an arterial stiffness index which serves as an arterial stiffness index of an organism; and a process of outputting the calculated arterial stiffness index to display means, by executing the first to fifth steps.

The PWVpcm2, which is a new arterial stiffness index, is obtained based on the highest blood pressure value Ps, the lowest blood pressure value Pd, and the pulse wave propagation velocity PWV′ before pressure calibration, through the execution of the first to fifth steps. This PWVpcm2 has a high primary correlation with PWVori which is an arterial stiffness index obtained by the PWV original method, which can reduce the risk of wrong diagnosis to be made by a doctor or a nurse even if they are accustomed to the PWVori. As compared with the conventional arterial stiffness index such as the pulse wave propagation velocity PWvori after pressure calibration obtained by the PWV original method or the CAVI, a value of an alteration coefficient (ac) is small, and a degree of substantial variation in data is extremely small. Thus, the present invention has an advantage that an inspection can be performed with high reliability, high stability, and high accuracy. Here, the ac is a numerical value which is a hundred multiple of a value obtained by dividing standard deviation by an average value, and equal to a statistics value generally called a coefficient of variation.

According to the arterial stiffness evaluation apparatus of the present invention, the arterial stiffness index calculating means calculates the PWvpcm1 value by executing, in place of the third to fifth steps, a sixth step of obtaining a PWVpcm1 value by substituting the highest blood pressure value Ps, the lowest blood pressure value Pd, and the pulse wave propagation velocity PWV′ before the pressure calibration, obtained from the pulse wave detecting means, the pulse wave propagation velocity deciding means, and the blood pressure detecting means, into the equation representing the PWVpcm1; and the degree of arterial stiffness is evaluated based on the PWVpcm1 value.

The PWVpcm1, which is a new arterial stiffness index, is obtained based on the highest blood pressure value Ps, the lowest blood pressure value Pd, and the pulse wave propagation velocity PWV′ before the pressure calibration, through the execution of the first, second, and sixth steps. This PWVpcm1 has a primary relation with the PWVori, which can reduce the risk of wrong diagnosis to be made by a doctor or a nurse even if they are accustomed to the PWVori.

The PWVori in the first step may also be obtained from the pulse wave propagation velocity PWV′ detected from carotid and tinea pulse waves. The use of the pulse wave propagation velocity PWV′ detected from the carotid and tinea pulse waves makes it possible to obtain an arterial stiffness index which reflects data of the pulse wave propagation velocity PWV′ based on a main artery which has conventionally been considered to be highly reliable.

The PWVori in the first step may also be obtained by pressure calibration with the lowest blood pressure value Pd set as 80 mmHg. As the PWVori is calibrated with the lowest blood pressure value pd of 80 mmHg, pressure calibration of an actually measured pulse wave propagation velocity PWV′ is facilitated, and which makes it possible to obtain a highly reliable pulse wave propagation velocity PWVori after pressure calibration.

According to the arterial stiffness evaluation apparatus of the present invention, the pulse wave detecting means may further include brachial pulse wave detecting means for detecting a pulse wave in the upper arm of an organism, and a popliteal pulse wave detecting means for detecting a pulse wave of poples of an organism. The use of pulse waves obtained in the upper arm and in the poples of the organism makes it possible to suppress a situation from being caused where vasospasm or vasoreflex is generated by fastening of an artery during actual measurement of the pulse wave propagation velocity PWV′, which attains more accurate arterial stiffness evaluation.

According to the arterial stiffness evaluation apparatus of the present invention, a pulse wave detector equipped with a distortion sensor may be used as the blood pressure detecting means. With the use of the pulse wave detector equipped with the distortion sensor as the blood pressure detecting means, the pulse wave can be directly converted into an electric signal and detected as a blood pressure, thereby making it possible to obtain an accurate blood pressure value. Accordingly, it is possible to prevent problems inherent in the conventional blood pressure measurement such as blood pressure measurement based on an oscillometric method where the detection result of a blood pressure is easy to be affected by external factors or where a blood pressure cannot be accurately measured depending on how the highest and lowest blood pressures are calculated.

According to the arterial stiffness evaluation apparatus of the present invention, based on the pulse wave detected by the pulse wave detector equipped with the distortion sensor, the highest blood pressure may be set as a blood pressure at a point of time when a pulse waveform having a negative notch unobserved in previous pulse waves but detected for the first time appears, and the lowest blood pressure is set as a blood pressure at a point of time when the notch disappears. The detection of the highest and lowest blood pressures based on the pulse wave makes it possible to determine the highest and lowest blood pressures easily and surely. Further, the values of the highest and lowest blood pressures are accurate.

Also, the present invention provides another arterial stiffness evaluation apparatus, including: pulse wave detecting means; pulse wave propagation velocity deciding means; blood pressure detecting means; and arterial stiffness index calculating means for calculating an arterial stiffness index to evaluate a degree of arterial stiffness based on a pulse wave propagation velocity and a blood pressure, in which the arterial stiffness index calculating means calculates at least one of an arterial stiffness index PWVpcm1 and an arterial stiffness index PWVpcm2 based on a highest blood pressure value Ps, a lowest blood pressure value Pd, and a pulse wave propagation velocity PWV′ before pressure calibration.

The arterial stiffness evaluation apparatus, which includes the arterial stiffness index calculating means for calculating at least one of the arterial stiffness indexes PWVpcm1 and PWVpcm2 based on the highest blood pressure value Ps, the lowest blood pressure value Ps, and the pulse wave propagation velocity PWV′ before the pressure calibration, is capable of evaluating arterial stiffness by using the PWVpcm1 or the PWVpcm2 which is a new arterial stiffness index.

Further, the present invention provides an arterial stiffness index calculation program which causes a computer to execute: a process of calculating an arterial stiffness index based on a highest blood pressure value Ps, a lowest blood pressure value Pd, and a pulse wave propagation velocity PWV′, based on an equation representing at least one of an arterial stiffness index PWVpcm1 and an arterial stiffness index PWVpcm2; and a process of outputting the arterial stiffness index thus calculated to display means.

The arterial stiffness index calculation program for causing the computer to execute: the process of calculating the arterial stiffness index based on the highest blood pressure Ps, the lowest blood pressure Pd, and the pulse wave propagation velocity PWV′, based on the equation representing at least one of the arterial stiffness indexes PWVpcm1 and PWVpcm2, and the process of outputting the calculated arterial stiffness index to the display means makes it possible to evaluate arterial stiffness by using the arterial stiffness index PWVpcm1 or PWVpcm2 which is a new arterial stiffness index.

According to the present invention, an accurate and universal arterial stiffness index can be obtained by measuring the highest blood pressure value Pd, the lowest blood pressure value Pd, and the pulse wave propagation velocity PWV′, without using any pressure-calibrated data prepared beforehand, and without using any special or expensive apparatus, thereby making it possible to diagnose a degree of arterial stiffness accurately and readily.

Further, according to the present invention, the new arterial stiffness index can be used in place of the pulse wave propagation velocity of the conventional PWV original method and the CAVI, the arterial stiffness index being low in variation, and the doctor or the nurse can correctly evaluate and diagnose a degree of arterial stiffness.

Contents of the present invention are not limited to the above. Other objects, advantages, features, and uses of the invention will become more apparent upon reading of the following description through accompanying drawings. It should be noted that proper changes in a scope not departing from the spirit of the invention are all within the invention.

### BRIEF DESCRIPTION OF THE DRAWINGS

In the accompanying drawings:

FIG. 1 is a block diagram showing an arterial stiffness evaluation apparatus according to an embodiment of the present invention;

FIG. 2 is a block diagram showing an arterial stiffness evaluation apparatus according to another embodiment of the present invention;

FIGS. 3A and 3B are diagrams showing a brachial pulse wave detector: FIG. 3A being a sectional diagram taken along the line SA-SB of FIG. 3B, and FIG. 3B being a plan diagram thereof;

FIG. 4 is an appearance diagram showing a state where the brachial pulse wave detector is fixed to an upper arm of an organism;

FIGS. 5A and 5B are diagrams showing a geniculate pulse wave detector: FIG. 5A being a sectional diagram taken along the line SB-SB of FIG. 5B, and FIG. 5B being a plan diagram thereof;

FIGS. 6A and 6B are diagrams showing a distortion sensor: FIG. 6B being a front diagram of a pressure transducer, and FIG. 6B being a plan diagram of the distortion sensor;

FIG. 7 is an amplifier block diagram showing processing of data obtained by the distortion sensor;

FIG. 8 is a time chart showing a pulse waveform detected from an organism by a pulse wave detector and other signals;

FIG. 9 is a time chart showing a waveform of a pulse wave detected by the distortion sensor;

FIG. 10 is a diagram showing a pulse wave propagation velocity calibration curve;

FIG. 11 is a graph showing a relation between a CAVI and a PWV;

FIG. 12 is a graph showing a relation between a PWVpcm1 and a PWV; and

FIG. 13 is a graph showing reproducibility of a PWVpcm2.

### DETAILED DESCRIPTION OF THE PREFERRED EMBODIMENTS

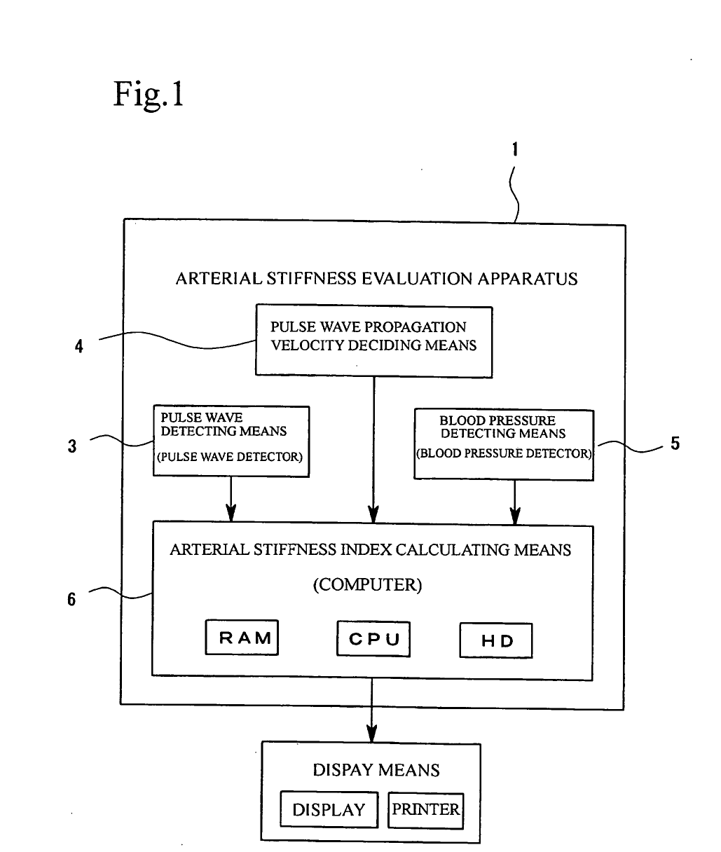

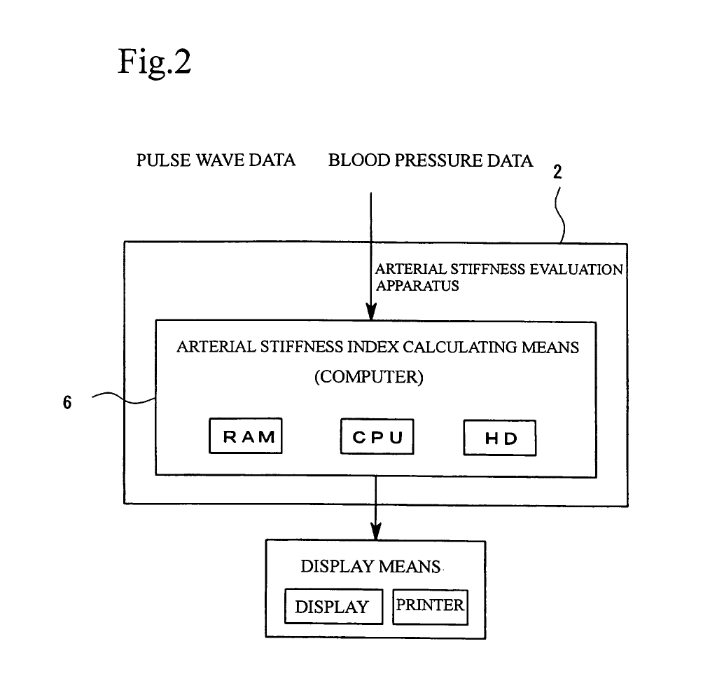

The preferred embodiments of the present invention will be described below in detail. An arterial stiffness evaluation apparatus of this embodiment includes pulse wave detecting means such as a pulse wave sensor, pulse wave propagation velocity deciding means for calculating a pulse wave propagation velocity from the detected pulse wave, a blood pressure detecting means such as a blood pressure gauge, and arterial stiffness index calculating means incorporated in a computer to calculate an arterial stiffness index from predetermined data. FIGS. 1 and 2 each show an example of the arterial stiffness evaluation apparatus.

Different modes of arterial stiffness evaluation apparatus 1 and 2 are shown in block diagrams of FIGS. 1 and 2. The arterial stiffness evaluation apparatus 1 shown in FIG. 1 includes pulse wave detecting means 3, pulse wave propagation velocity deciding means 4, blood pressure detecting means 5, and arterial stiffness index calculating means 6. The arterial stiffness index calculating means 6 is configured by including a computer incorporating a central processing unit (CPU), a random access memory (RAM), a hard disk drive (HDD), and the like, and a computer program to operate the computer. For example, when an arterial stiffness index calculation program recorded in an external recording medium such as a CD-ROM is read into the RAM and executed by the CPU, pulse wave data obtained from the pulse wave detecting means 3, blood pressure data of a highest/lowest blood pressure obtained from the blood pressure detecting means 5, and data such as a predetermined blood vessel length input from an outside are substituted into a predetermined arithmetic equation to calculate an arterial stiffness index. The calculated arterial stiffness index is displayed together with a patient name and past data in a display or a printer and output to be used.

A function of the arterial stiffness evaluation apparatus 2 shown in FIG. 2 is substantially similar to that of the arterial stiffness evaluation apparatus shown in FIG. 1. However, instead of using the data obtained from the pulse wave detecting means 3 or the blood pressure detecting means 5, existing pulse wave data and blood pressure data are input to the computer as the arterial stiffness index calculating means 6 to be used. Thus, past data can be used in the arterial stiffness evaluation apparatus 2.

The pulse wave detecting means 3, the pulse wave propagation velocity deciding means 4, the blood pressure detecting means 5, and the arterial stiffness index calculating means 6 will be described below in more detail.

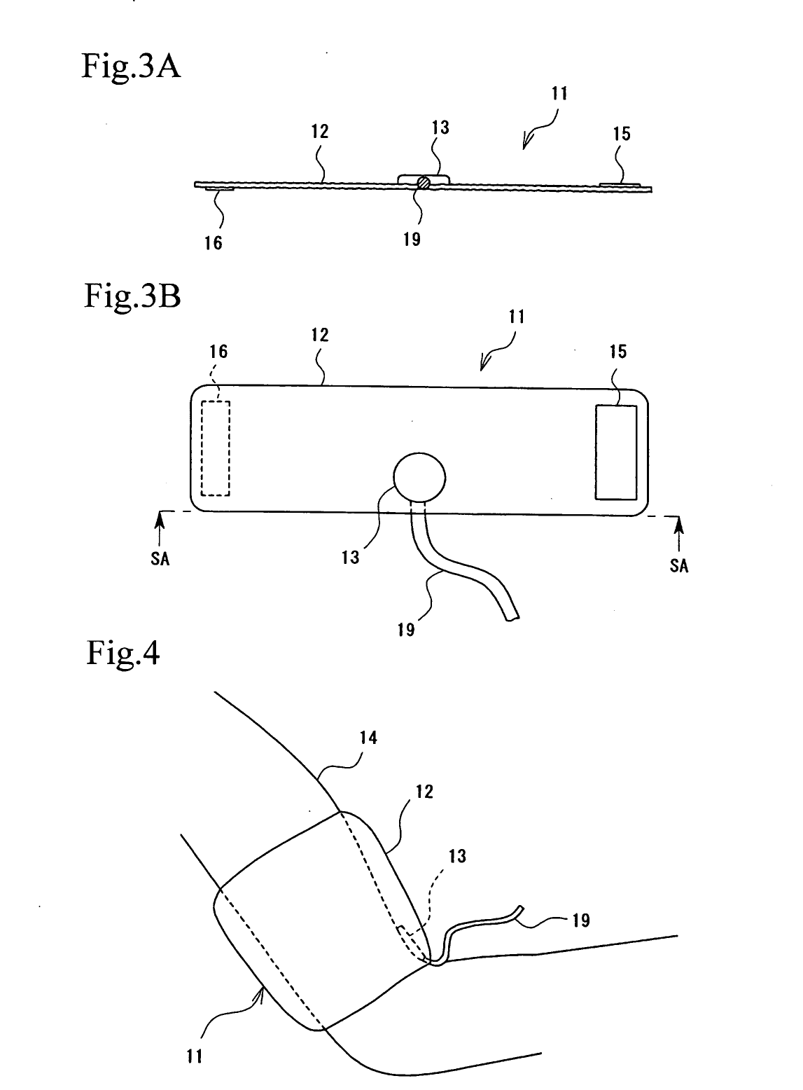

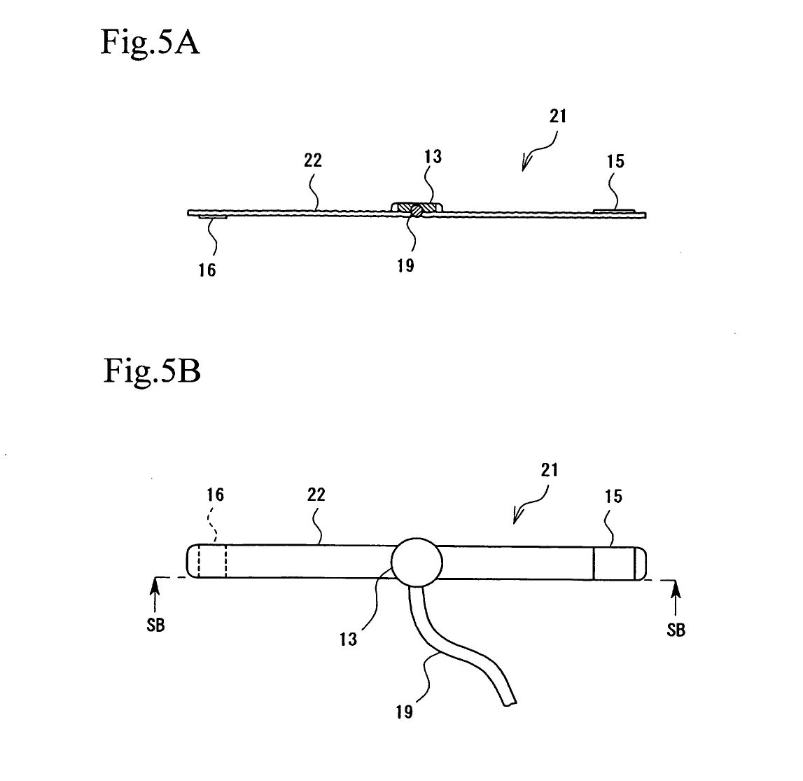

Pulse Wave Detecting Means: The pulse wave detecting means 3 can include a brachial pulse wave detector 11 of FIGS. 3A and 3B for detecting a pulse wave from an upper arm of an organism, and a popliteal pulse wave detector 21 of FIG. 5 for detecting a pulse wave from a poples of the organism. Those pulse wave detectors 11 and 21 can detect pulse waves from the organism and output pulse waveforms to the display. The brachial pulse wave detector 11 has a distortion sensor 13 mounted to a strip-shaped cuff 12 at a portion which is an end in a short direction and a center in a longitudinal direction. As shown in FIG. 4, the cuff 12 to which the distortion sensor 13 has been mounted is wound on an upper arm 14 and fixed to the arm with, for example, magic tapes (registered trademark) 15 and 16 stitched to the cuff 12, whereby the distortion sensor 13 can be pressed and fixed by low pressure of about 10 mmHg in its abutted state on a top of a brachial artery beat part. As the distortion sensor 13 disposed in the brachial pulse wave detector 11 is highly sensitive, a pulse wave can be correctly detected even if slight positional shifting occurs.

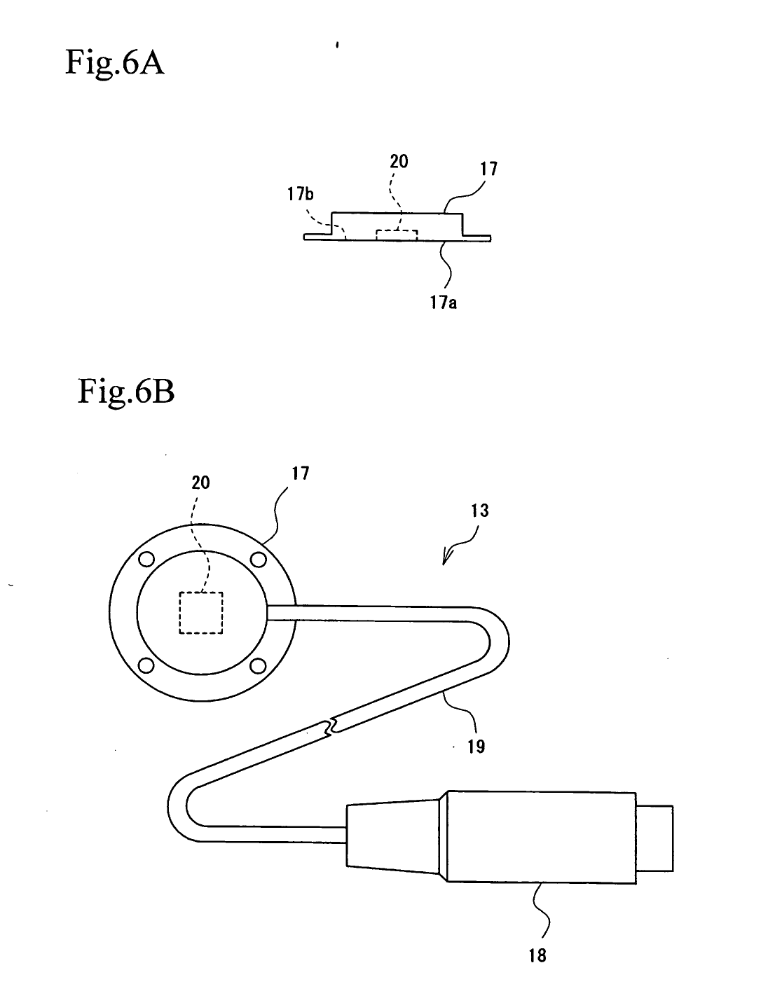

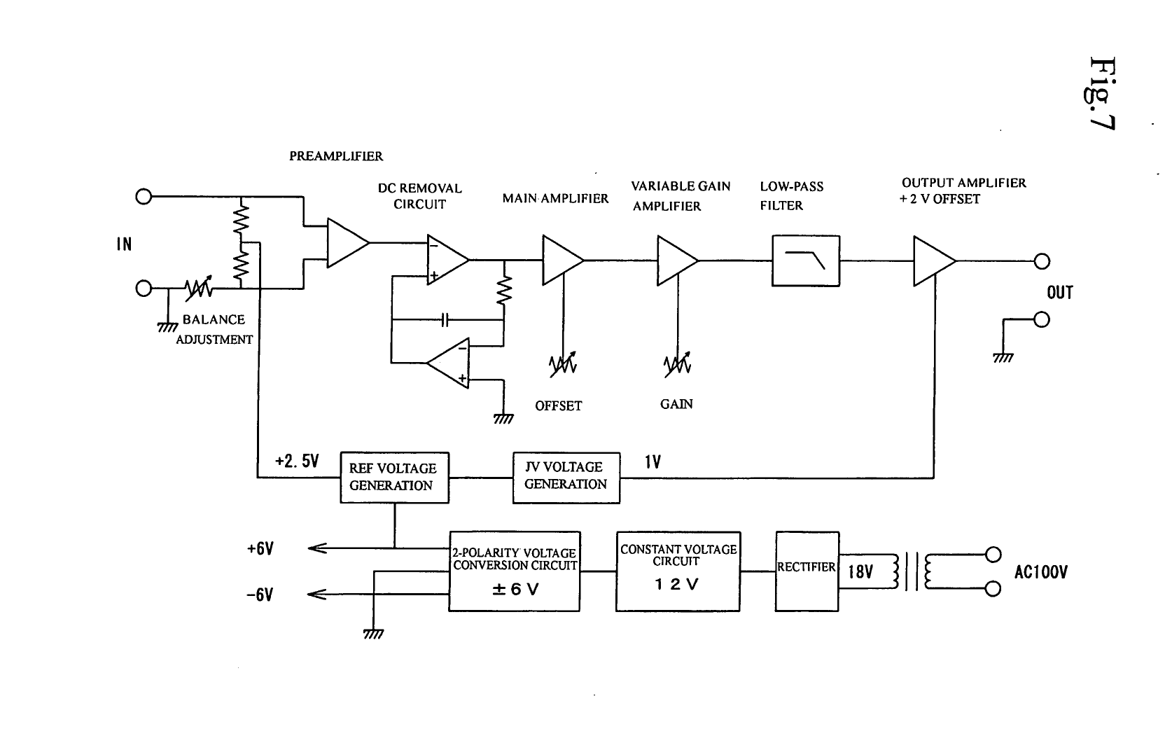

FIGS. 6A and 6B show an appearance of the distortion sensor 13. For example, the distortion sensor 13 is configured in such a manner that a pressure transducer 17 having a cylindrical or hat outer shape of a diameter of about 30 mm and a thickness of about 5 mm to 20 mm is connected to a mini-DIN plug (4P) 18 connected to an amplifier (not shown) through a cord 19, and a semiconductor strain gauge 20 is disposed in a backside 17b such as a stainless plate appearing in a front surface 17a of the pressure transducer 17. When the distortion sensor 13 receives pressure (pulse pressure) from an organism, distortion occurs in the semiconductor strain gauge 20, and the distortion is converted into an electric signal, and amplified by an amplifier (not shown) which is a part of the pulse wave detecting means to be detected (FIG. 7).

Different from a cuff used for a blood pressure gauge using an oscillometric method, the cuff 12 used in the brachial pulse wave detector 11 does not press a pulse wave detecting part to stop a blood flow but only needs to fix the distortion sensor 13 so as not to move during pulse wave detection. However, as the brachial pulse wave detector 11 preferably functions also as blood pressure detecting means 5, the cuff 12 can be adapted to apply a pressing force on a measuring part, thereby stopping a blood flow.

The popliteal pulse wave detector 21 may be identical to the brachial pulse wave detector 11. However, measurement of blood pressure from the poples is not required or mental and physical loads are imposed on a test subject if the cuff is wound on a thigh, as shown in FIGS. 5A and 5B, so the distortion sensor 13 should preferably be mounted to a strip-shaped binder 22 such as a thin magic band (registered trademark) in place of the cuff. For the strip-shaped binder 22, a binder made of cloth or a binder which is made elastic by mixing rubber in cloth can be used in addition to the magic band (registered trademark), and magic tapes (registered trademark) 15 and 16 should preferably be used for the binding part. An annular fastener having no binding part may be used as long as elasticity is large. However, excessive fastening of the poples exceeding 30 mmHg must be prevented. Further, a pulse wave detector can be configured in such a manner that the pressure transducer 17 is fixed to the measuring part only by an adhesive tape or the like without using any strip-shaped binders 12 or 22. In this case, almost no pressure should preferably be applied to the measuring part.

In addition to the sensor equipped with the distortion sensor 13, the pulse wave detector can use, as pulse wave detecting sensor 3, various well-known pulse wave sensors such as a carotid pulse wave sensor for detecting a carotid pulse wave generated from a carotid artery, a tinea pulse wave sensor for detecting a tinea pulse wave generated from a tinea artery by being fitted to an inguinal part, a heart sound sensor for detecting a heart sound generated from the heart by being fitted directly above a heart, a pressure sensor connected to a cuff to be wound on an ankle or the upper arm. An electrocardiographic guidance system having a plurality of electrodes to be fixed to both wrists to obtain an electrocardiographic waveform may be disposed.

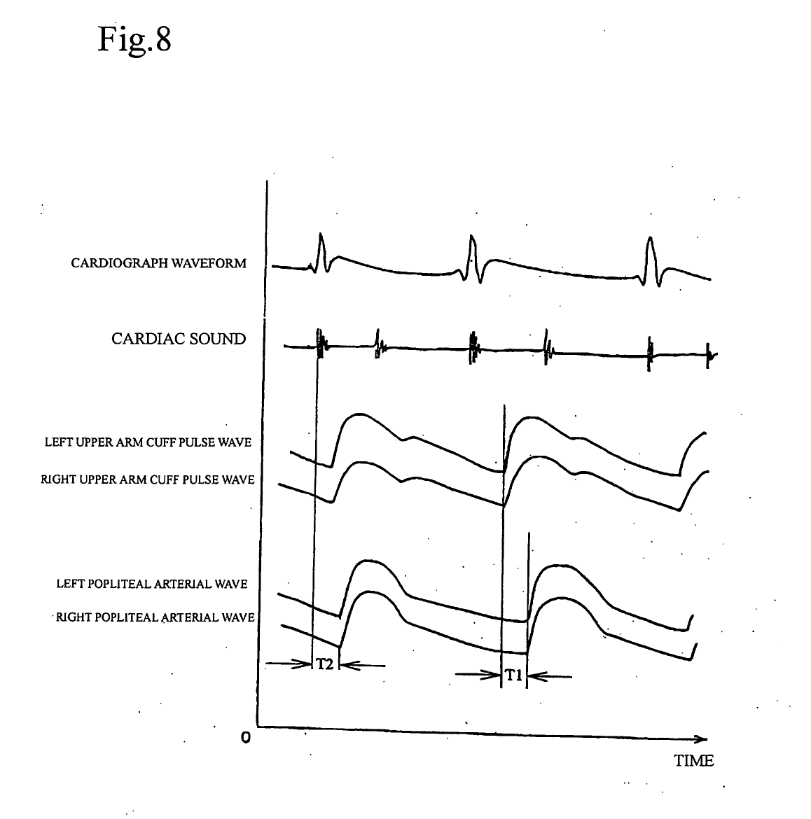

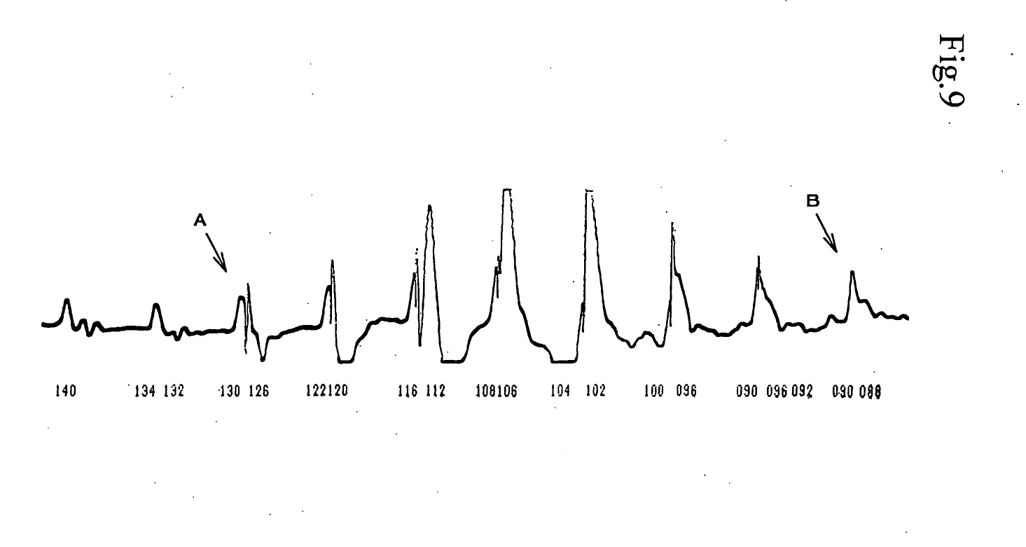

Pulse Wave Propagation Velocity Deciding Means: The pulse wave propagation velocity deciding means 4 divides a length of an artery between two points of the organism whose pulse wave has been detected by pulse wave propagation time to obtain a pulse wave propagation velocity. FIG. 8 is a time chart showing pulse waves, an electrocardiographic waveform, and a heart sound detected from various pulse wave detecting means 3 along a common time axis. For example, pulse wave propagation time from the upper arm to the poples becomes a difference between pulse wave propagation time from a main artery valve port to the poples and pulse wave propagation time from the main artery to the upper part. When this time is assumed to be T1, a distance (L1−L2) between pulse wave detecting parts is divided by the pulse wave propagation time (T1) to obtain a pulse wave propagation velocity, i.e., (L1−L2)/T1.

Pulse wave propagation time T2 from the main artery valve port to the poples is measured by using a heart sound or the like obtained from a heart sound sensor for detecting a heart sound in place of using the brachial pulse wave detector 11. Accordingly, a pulse wave propagation velocity can be obtained by dividing the distance L1 by the time T2. This pulse wave propagation velocity detecting means 4 can be configured by including a computer incorporating a central processing unit (CPU), a random access memory (RAM), a hard disk drive (HDD), and the like, and a computer program for boosting the computer. The computer incorporated in the pulse wave detecting means 3, which is also composed of a CPU, a RAM, an HDD, etc., can be used as pulse wave propagation velocity deciding means, or the computer of the arterial stiffness index calculating means 6 can be used as pulse wave propagation velocity deciding means.

Blood Pressure Detecting Means: The blood pressure detecting means 5 detects highest and lowest blood pressures of the organism among blood pressures. A well-known blood pressure gauge can generally be used. However, the blood pressure gauge should preferably include the distortion sensor 13 to obtain an accurate blood pressure value. While the brachial pulse wave detector 11 can be used as the blood pressure detecting means 5, a highest blood pressure value Ps and a lowest blood pressure Pd are detected based on a change in pulse waveform when a measuring part is pressed, an artery is closed, and then pressure is gradually reduced. Thus, the cuff to be simply wound on the arm or the leg is not sufficient. A cuff as a pressing band which can slow a blood flow by pressing the arm or the leg must be used. For such the cuff, a cuff used in the blood pressure gauge based on the oscillometric method can be utilized.

Detection of highest and lowest blood pressures by the pulse wave detector equipped with the distortion sensor 13 is performed based on a change state where a pulse waveform detected through the distortion sensor 13 is changed after the artery is opened. FIG. 9 shows a pulse waveform generated in the process of changing a pressing of the cuff wound on the upper arm. The pulse waveform shown in the figure is based on a pulse wave detected in the process of reducing cuff pressure, and it can be understood that the waveform changes in the process of reducing the cuff pressure. In this pulse waveform, a highest blood pressure is at a point of appearance time of a waveform where a negative notch unobserved in previous waveforms is detected as a waveform precomponent for the first time, and a lowest blood pressure is a blood pressure value at a point of appearance time of a waveform where the notch has disappeared. Accordingly, the highest and lowest blood pressures are detected based on the appearance/disappearance of the negative notch in the pulse waveform, and the highest and lowest blood pressures can be easily decided. It has been proven that the highest and lowest blood pressures obtained by this method are matched with those measured by using an invasive method having a catheter inserted into a radial artery to be accurate in values.

As described above, in addition to the case of using the pulse wave detecting means using the distortion sensor 13 for the blood pressure detecting means 5, various blood pressure gauges utilizing the oscillometric method and Korotkoff sound detection can be used for the blood pressure detecting means 5. However, the employment of the pulse wave detecting means using the distortion sensor 13 is preferable in that accurate highest and lowest blood pressures can be detected.

In the case of blood pressure detection by the blood pressure detecting means 5, even if vasoreflex or vasospasm occurs, a detection result should preferably be prevented from being reflected in a pulse wave propagation velocity. Hence, pulse wave detection should preferably be carried out before the detection of highest and lowest blood pressures.

Arterial stiffness index Calculating Means: The arterial stiffness index calculating means obtains PWVpcm1 and PWVpcm2 which become new indexes to evaluate arterioschlerosis by executing the following steps.

First Step: The number enough to obtain a statistically significant regression equation is sampled from a data group of a highest blood pressure value Ps, a lowest blood pressure value pd, a pulse wave propagation velocity PWV′, and the like which are obtained from many test subjects, and a pulse wave propagation velocity PWVori after pressure calibration, and CAVI are calculated from these data. For example, PWVori and CAVI are calculated from Ps, Pd, and PWV′ of a test subject a1 measured on June 1. Specifically, pressure is calibrated from PWV's before pressure calibration to derive a PWVori value, and the Ps, Pd, and the PWV are substituted into the equation of CAVI=ln(Ps/Pd)×PWV² to derive a CAVI value. Similarly, PWVori and CAVI are calculated from Ps, Pd, and PWV of the test subject a1 measured on December 20, PWVori and CAVI are calculated from Ps, Pd, and PWV′ of a test subject a2 measured on July 8, . . . and PWVori and CAVI are calculated from Ps, Pd, and PWV′ of a test subject an measured on day/month.

In general, because of a relation where a blood pressure increase is accompanied by an increase in pulse wave propagation velocity, pulse wave propagation velocities in various blood pressure values are affected by blood pressures, and their numerical values themselves cannot simply be compared with one another. Accordingly, pulse wave propagation velocities PWV′ in various blood pressure values must be converted into pulse wave propagation velocities PWV in a predetermined blood pressure value. A pulse wave propagation velocity after calibration based on the blood pressure value is a pulse wave propagation velocity PWVori after pressure calibration.

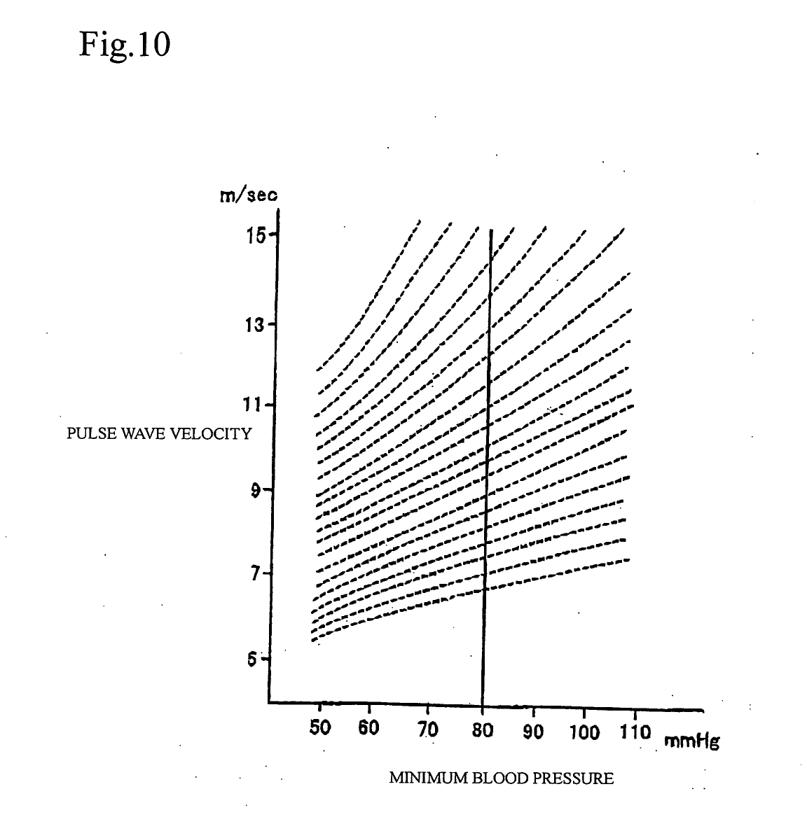

As a method of calibrating the pulse wave propagation velocity PWV′ by a blood pressure, a method of obtaining a pulse wave velocity calibration curve is available. According to this method, many cases are statistically analyzed to create a pulse wave velocity calibration curve similar to that of FIG. 10 indicating a relation between a lowest blood pressure (in FIG. 10, represented as “minimum blood pressure”) and a pulse wave propagation velocity (in FIG. 10, represented as “pulse wave velocity”), and a pulse wave propagation velocity actually measured under any blood pressure value is converted into a pulse wave propagation velocity at a minimum blood pressure of 80 mmHg according to this pulse wave velocity calibration curve. As another pressure calibration method, there is a method of obtaining PWVori based on an equation of PWVori=PWVpd×(1/√pd/√80) (PWVpd indicates a pulse wave propagation velocity in the case of minimum blood pressure Pd, Pd indicates a lowest blood pressure, √Pd indicts a square root of Pd, and √80 indicates a square root of 80). By any one of those methods, it is possible to obtain a pulse wave propagation velocity PWVori after pressure calibration. When a PWVori value is stored as data from an existing measuring result in the process of obtaining PWVori, the PWVori can be used.

Next, a regression equation representing the PWVori by a quadratic equation of the CAVI is derived from a relation between the PWVori and the CAVI. The description of the above-mentioned example will continue. Values of the PWVori and the CAVI which have been obtained based on the data of the test subject a1 measured on June 1 are plotted in a graph where an ordinate indicates PWVori and an abscissa indicates CAVI. Similarly, a PWVori value and a CAVI value which have been obtained from the data of the test subject a1 measured on December 20 are plotted, a PWVori value and a CAVI value which have been obtained from the data of the test subject a2 measured on July 8 are plotted, . . . , and a PWVori value and a CAVI value which have been obtained from the data of the test subject an measured on day/month are plotted. From a distribution of points thus obtained, a regression equation represented as follows is derived:
PWVori=A(CAVI)²+B(CAVI)+C (2)
(where A, B, and C are constants).
In other words, the PWVori is represented by a quadratic equation where the CAVI is a variable, and a relation between the PWVori and the CAVI is represented.

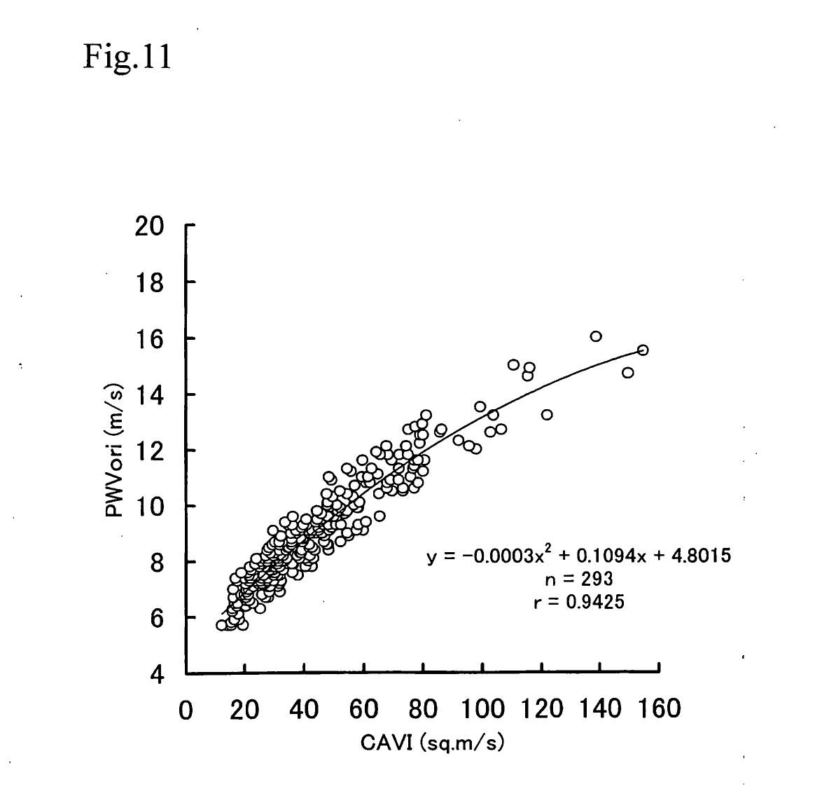

This is shown more specifically in FIG. 11. In FIG. 11, a PWVori value and a CAVI value are calculated from data of examples of 293 patients in total such as pulse wave propagation velocities PWV′ calculated based on pulse waves measured from highest blood pressures Pd and lowest blood pressures Pd measured from upper arms of the patients, tinea pulse waves, carotid pulse waves, and heart sounds, the PWVori values are plotted in an ordinate, and the CAVI values are plotted in an abscissa. When a regression equation representing PWVori by a quadratic equation of CAVI is derived from distributed states of both PWVori and CAVI values, it can be represented by the following equation (3):
y=−0.0003x²+0.1094x+4.8015 (3)
where y is PWVori and x is CAVI. The first step has been described.

Second Step: The regression equation obtained in the first step is used as an equation to represent PWVpcm1. In other words, with the coefficients of the regression equation (2) maintained as they are, as an equation to represent PWVpcm1 by CAVI, the following equation (4) is obtained:
PWVpcm1=A(CAVI)²+B(CAVI)+C (4)
Accordingly, PWVpcm which is a new arterial stiffness index calibrated by CAVIPWVori as one of arterial stiffness indexes is obtained.

When applied to the example shown in FIG. 11, the following equation (5) is obtained:
PWVpcm1=−0.0003(CAVI)²+0.1094(CAVI)+4.8015 (5)

Third Step: This time, each CAVI value is substituted into the equation representing PWVpcm1 obtained in the second step to obtain each PWVpcm1 value based on each CAVI value. Then, a regression equation representing PWVori by a linear equation of the PWVpcm1 is derived from a relation between each PWvori value and the PWVpcm1 value obtained here. In other words, each PWvori value is plotted in an ordinate, each PWVpcm1 is plotted in an abscissa, and a regression equation representing PWVori by PWVpcm is derived from distributed states of both thereof. This regression equation is represented as follows:
PWVori=E(PWVpcm1)+F (6)
(where E and F are constants).

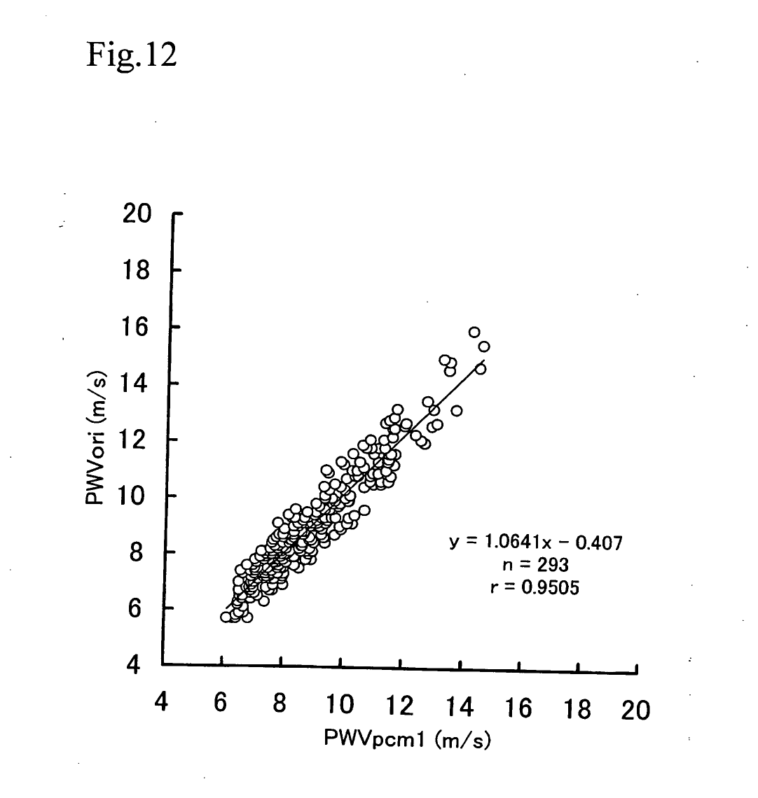

FIG. 12 shows a relation between PWVori obtained based on data identical to that shown in FIG. 11 and the PWVpcm1 obtained by the above-mentioned method. In FIG. 12, as in the case of FIG. 11, PWVori is plotted in an ordinate, and PWVpcm1 is plotted in an abscissa. When a regression equation representing PWvori by a linear equation of PWVpcm1 is derived from a relation between the PWVori and the PWVpcm1 shown in FIG. 12, the following equation (7) is established:
y=1.0641x−0.407 (7)
where y is PWVori and x is PWVpcm1. The third step has been described.

Fourth Step: The regression equation (6) obtained in the third step is set as an equation representing PWVpcm2 which becomes a new arterial stiffness index. With the coefficients of the regression equation (6) maintained as they are, an equation representing PWVpcm2 by PWVpcm1 is set. Thus, the following equation (8) is obtained:
PWVpcm2=E(PWVpcm1)+F (8)
(where E and F are constants).

When applied to the example shown in FIG. 12, the following equation (9) is established:
PWVpcm2=1.0641(PWVpcm1)−0.407 (9)

When the equation (4) is substituted into the equation (8) which represents PWVpcm2 to delete PWVpcm1,
PWVpcm2=E(PWVpcm1)+F (8)
PWVpcm1=A(CAVI)²+B(CAVI)+C (4),
the following equation (9) is obtained:
PWVpcm2=E{A(CAVI)²+B(CAVI)+C}+F (9).
Accordingly, when the CAVI is represented by a highest blood pressure Ps, a lowest blood pressure Pd, and a pulse wave propagation velocity PWV′, the following equation (10) of representing PWVpcm2 is obtained:
PWVpcm2=A·E·(ln(Ps/Pd)×PWV′²)²+B·E·(ln(Ps/Pd)×PWV′²)+C·E+F (10)
(where A, B, C, E, and F are constants).

When calculation is executed based on data shown in FIGS. 12 and 11, the equation (5) is substituted into the equation (9) to set the following equation (11):
PWVpcm2=1.0641{−0.0003(CAVI)²+0.1094(CAVI)+4.8015}−0.407 (11),
thereby obtaining the following equation (12)
PWVpcm2=−0.0003192(CAVI)²+0.1164(CAVI)+4.7023 (12).
Because of CAVI=ln(Ps/Pd)×PWV′², the following equation (13) is established:
PWVpcm2=−0.0003192(ln(Ps/Pd)×PWV′²)²+0.1164(ln(Ps/Pd)×PWV′2)+4.7023 (13).

The PWVpcm2 thus obtained can be used as a new index of arterial stiffness. Accordingly, by substituting Ps, Pd, and PWV′ actually measured from a new test subject into the equation (13), an arterial stiffness index PWVpcm2 is obtained.

The first to fifth steps have been executed to obtain the PWVpcm2 which is a new arterial stiffness index. As described above, the PWVpcm1 obtained by executing the first and second steps can be used as an arterial stiffness index.

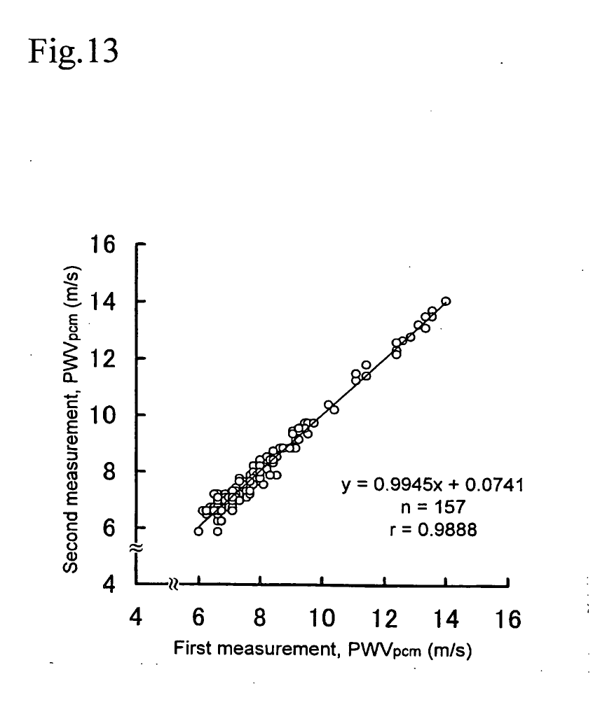

A graph of FIG. 13 shows reproducibility of PWVpcm2 (in FIG. 13, represented as “PWVpcm”) obtained as a result of measuring Ps, Pd, and PWV′ of totally 157 test subjects, specifically, 105 males and 52 females, aged 24 to 81, including outpatients, inpatients, and healthy volunteers of SK hospital twice at an interval of several days. A numerical value range of PWVpcm2 is about 6 to 14 m/s, a regression equation is y=0.9945x+0.0741, and a correlation coefficient is r=0.9888. It can be understood from this that highly accurate reproducibility is obtained irrespective of high/low values of PWVpcm2.

Tables 1 to 3 show results of arbitrarily extracting 12 examples from 157 targets of FIG. 13, and measuring and detecting various factors such as Ps, Pd, and PWV′ five to six times (average 5.2 times) for each test subject for 2 weeks. For each factor, an average value x, standard deviation SD, SD √x, ac are obtained. A range of ac of PWVpcm2 of 12 examples is 6 to 14 m/s, and an average of PWVpcm2 of 12 examples is 2.6±0.67% indicating high accuracy. On the other hand, an average of ac of Ps of 12 examples is 7.3±2.38%, and an average of ac of Pd is 6.5±2.74%.

TABLE 1

| No. | | | | | | | | | | | |
|---|---|---|---|---|---|---|---|---|---|---|---|
| Age | | Number of measurements | | | | | | | | | |
| Sex | Factors | 1 | 2 | 3 | 4 | 5 | 6 | x̅ | SD | SD/ x̅ | ac. |
| No. 3 | Ps | 152 | 181 | 136 | 156 | 140 | | 153.0 | 17.7 | 0.116 | 11.6 |
| 49 y | Pd | 101 | 119 | 89 | 107 | 88 | | 100.8 | 13.0 | 0.129 | 12.9 |
| M | In Ps/Pd | 0.41 | 0.42 | 0.42 | 0.38 | 0.46 | | 0.4 | 0.03 | 0.075 | 7.5 |
| | PWV′ | 7.6 | 7.0 | 7.3 | 7.7 | 6.9 | | 7.3 | 0.4 | 0.049 | 4.9 |
| | PWV′² | 57.52 | 48.93 | 53.25 | 59.51 | 47.44 | | 53.3 | 5.2 | 0.098 | 9.8 |
| | PWV′²/Pd | 0.6 | 0.4 | 0.6 | 0.6 | 0.5 | | 0.5 | 0.1 | 0.135 | 13.5 |
| | PWV ori | 6.7 | 5.7 | 6.9 | 6.7 | 6.6 | | 6.5 | 0.5 | 0.071 | 7.1 |
| | CAVI | 7.2 | 6.8 | 7.1 | 7.1 | 7.0 | | 7.0 | 0.2 | 0.022 | 2.2 |
| | PWV pcm | 7.3 | 7.0 | 7.2 | 7.2 | 7.1 | | 7.1 | 0.1 | 0.016 | 1.6 |
| No. 5 | Ps | 129 | 113 | 132 | 130 | 128 | 135 | 127.8 | 7.7 | 0.060 | 6.0 |
| 38 y | Pd | 95 | 79 | 90 | 90 | 92 | 96 | 90.3 | 6.1 | 0.067 | 6.7 |
| F | In Ps/Pd | 0.31 | 0.36 | 0.38 | 0.37 | 0.33 | 0.34 | 0.3 | 0.03 | 0.080 | 8.0 |
| | PWV′ | 6.8 | 7.1 | 7.0 | 7.0 | 6.7 | 7.7 | 7.1 | 0.3 | 0.049 | 4.9 |
| | PWV′² | 45.91 | 50.21 | 49.64 | 48.53 | 45.42 | 59.32 | 49.8 | 5.0 | 0.101 | 10.1 |
| | PWV′²/Pd | 0.5 | 0.6 | 0.6 | 0.5 | 0.5 | 0.6 | 0.6 | 0.1 | 0.113 | 11.3 |
| | PWV ori | 6.2 | 7.1 | 6.6 | 6.6 | 6.3 | 7.0 | 6.6 | 0.4 | 0.056 | 5.6 |
| | CAVI | 6.3 | 6.6 | 6.7 | 6.6 | 6.5 | 6.8 | 6.6 | 0.2 | 0.026 | 2.6 |
| | PWV pcm | 6.3 | 6.7 | 6.8 | 6.7 | 6.4 | 6.9 | 6.6 | 0.3 | 0.038 | 3.8 |
| No. 7 | Ps | 166 | 164 | 181 | 143 | 149 | | 160.6 | 15.0 | 0.093 | 9.3 |
| 59 y | Pd | 104 | 103 | 113 | 89 | 94 | | 100.6 | 9.3 | 0.093 | 9.3 |
| M | In Ps/Pd | 0.47 | 0.47 | 0.47 | 0.47 | 0.46 | | 0.5 | 0.01 | 0.011 | 1.1 |
| | PWV′ | 9.6 | 9.6 | 8.8 | 9.4 | 9.2 | | 9.3 | 0.3 | 0.036 | 3.6 |
| | PWV′² | 93.08 | 93.08 | 78.12 | 88.05 | 84.54 | | 87.4 | 6.3 | 0.072 | 7.2 |
| | PWV′²/Pd | 0.9 | 0.9 | 0.7 | 1.0 | 0.9 | | 0.9 | 0.1 | 0.126 | 12.6 |
| | PWV ori | 8.5 | 8.5 | 7.4 | 8.9 | 8.5 | | 8.4 | 0.5 | 0.065 | 6.5 |
| | CAVI | 8.9 | 8.9 | 8.3 | 8.8 | 8.6 | | 8.7 | 0.3 | 0.029 | 2.9 |
| | PWV pcm | 9.2 | 9.1 | 8.6 | 9.0 | 8.8 | | 8.9 | 0.3 | 0.030 | 3.0 |
| No. 8 | Ps | 133 | 144 | 158 | 161 | 148 | | 148.8 | 11.3 | 0.076 | 7.6 |
| 65 y | Pd | 87 | 104 | 97 | 101 | 94 | | 96.6 | 6.6 | 0.068 | 6.8 |
| M | In Ps/Pd | 0.42 | 0.33 | 0.49 | 0.47 | 0.45 | | 0.4 | 0.1 | 0.147 | 14.7 |
| | PWV′ | 9.0 | 9.7 | 8.6 | 8.6 | 8.7 | | 8.9 | 0.5 | 0.054 | 5.4 |
| | PWV′² | 81.24 | 94.41 | 73.32 | 73.32 | 76.15 | | 79.7 | 8.8 | 0.111 | 11.1 |
| | PWV′²/Pd | 0.9 | 0.9 | 0.8 | 0.7 | 0.8 | | 0.8 | 0.1 | 0.111 | 11.1 |
| | PWV ori | 8.6 | 8.5 | 7.8 | 7.6 | 8.1 | | 8.1 | 0.4 | 0.055 | 5.5 |
| | CAVI | 8.2 | 7.8 | 8.3 | 8.2 | 8.1 | | 8.1 | 0.2 | 0.024 | 2.4 |
| | PWV pcm | 8.3 | 8.0 | 8.5 | 8.3 | 8.3 | | 8.3 | 0.2 | 0.022 | 2.2 |

TABLE 2

| No. | | | | | | | | | | | |
|---|---|---|---|---|---|---|---|---|---|---|---|
| Age | | Number of measurements | | | | | | | | | |
| Sex | Factors | 1 | 2 | 3 | 4 | 5 | 6 | x̅ | SD | SD/ x̅ | ac. |
| No. 9 | Ps | 111 | 123 | 117 | 118 | 105 | 98 | 112.0 | 9.3 | 0.083 | 8.3 |
| 24 y | Pd | 60 | 67 | 61 | 59 | 63 | 55 | 60.8 | 4.0 | 0.066 | 6.6 |
| M | In Ps/Pd | 0.62 | 0.61 | 0.65 | 0.69 | 0.51 | 0.58 | 0.6 | 0.1 | 0.103 | 10.3 |
| | PWV′ | 6.7 | 7.0 | 6.4 | 6.2 | 7.1 | 6.7 | 6.7 | 0.3 | 0.049 | 4.9 |
| | PWV′² | 45.53 | 49.28 | 41.03 | 38.85 | 49.78 | 45.09 | 44.9 | 4.4 | 0.097 | 9.7 |
| | PWV′²/Pd | 0.8 | 0.7 | 0.7 | 0.7 | 0.8 | 0.8 | 0.7 | 0.1 | 0.086 | 8.6 |
| | PWV ori | 7.8 | 7.7 | 7.3 | 7.3 | 8.0 | 8.1 | 7.7 | 0.3 | 0.043 | 4.3 |
| | CAVI | 7.5 | 7.7 | 7.5 | 7.3 | 7.4 | 7.3 | 7.5 | 0.2 | 0.020 | 2.0 |
| | PWV pcm | 7.7 | 7.9 | 7.6 | 7.6 | 7.5 | 7.5 | 7.6 | 0.2 | 0.021 | 2.1 |
| No. 40 | Ps | 168 | 168 | 186 | 178 | 155 | | 171.0 | 11.7 | 0.068 | 6.8 |
| 79 y | Pd | 89 | 85 | 97 | 99 | 90 | | 92.0 | 5.8 | 0.063 | 6.3 |
| M | In Ps/Pd | 0.64 | 0.68 | 0.65 | 0.59 | 0.54 | | 0.6 | 0.1 | 0.088 | 8.8 |
| | PWV′ | 8.8 | 8.4 | 8.1 | 8.1 | 8.5 | | 8.4 | 0.3 | 0.034 | 3.4 |
| | PWV′² | 77.30 | 70.52 | 66.21 | 65.39 | 72.36 | | 70.4 | 4.9 | 0.069 | 6.9 |
| | PWV′²/Pd | 0.9 | 0.8 | 0.7 | 0.7 | 0.8 | | 0.8 | 0.1 | 0.120 | 12.0 |
| | PWV ori | 8.3 | 8.1 | 7.4 | 7.3 | 8.0 | | 7.8 | 0.5 | 0.061 | 6.1 |
| | CAVI | 9.3 | 9.3 | 9.1 | 8.6 | 8.4 | | 8.9 | 0.4 | 0.047 | 4.7 |
| | PWV pcm | 9.6 | 9.6 | 9.1 | 8.7 | 8.8 | | 9.2 | 0.4 | 0.047 | 4.7 |
| No. 43 | Ps | 120 | 110 | 115 | 129 | 117 | | 118.2 | 7.0 | 0.060 | 6.0 |
| 33 y | Pd | 67 | 60 | 62 | 68 | 63 | | 64.0 | 3.4 | 0.053 | 5.3 |
| M | In Ps/Pd | 0.58 | 0.61 | 0.62 | 0.64 | 0.62 | | 0.6 | 0.02 | 0.034 | 3.4 |
| | PWV′ | 6.8 | 6.7 | 6.8 | 6.9 | 6.9 | | 6.8 | 0.1 | 0.011 | 1.1 |
| | PWV′² | 46.46 | 45.10 | 46.00 | 47.88 | 46.92 | | 46.5 | 1.0 | 0.022 | 2.2 |
| | PWV′²/Pd | 0.7 | 0.8 | 0.7 | 0.7 | 0.7 | | 0.7 | 0.03 | 0.036 | 3.6 |
| | PWV ori | 7.4 | 7.8 | 7.7 | 7.5 | 7.7 | | 7.6 | 0.1 | 0.018 | 1.8 |
| | CAVI | 7.5 | 7.5 | 7.6 | 7.8 | 7.7 | | 7.6 | 0.1 | 0.017 | 1.7 |
| | PWV pcm | 7.6 | 7.6 | 7.8 | 8.0 | 7.8 | | 7.8 | 0.1 | 0.018 | 1.8 |
| No. 44 | Ps | 123 | 118 | 112 | 122 | 130 | | 121.0 | 6.6 | 0.055 | 5.5 |
| 56 y | Pd | 83 | 84 | 78 | 84 | 88 | | 83.4 | 3.6 | 0.043 | 4.3 |
| M | In Ps/Pd | 0.39 | 0.34 | 0.36 | 0.37 | 0.39 | | 0.4 | 0.02 | 0.059 | 5.9 |
| | PWV′ | 7.3 | 7.0 | 6.9 | 7.2 | 6.9 | | 7.1 | 0.2 | 0.022 | 2.2 |
| | PWV′² | 52.82 | 49.52 | 47.98 | 51.68 | 47.98 | | 50.0 | 2.2 | 0.044 | 4.4 |
| | PWV′²/Pd | 0.6 | 0.6 | 0.6 | 0.6 | 0.5 | | 0.6 | 0.03 | 0.058 | 5.8 |
| | PWV ori | 7.1 | 6.9 | 7.0 | 7.0 | 6.6 | | 6.9 | 0.2 | 0.029 | 2.9 |
| | CAVI | 7.0 | 6.6 | 6.6 | 6.5 | 6.9 | | 6.7 | 0.2 | 0.032 | 3.2 |
| | PWV pcm | 7.0 | 6.6 | 6.6 | 6.8 | 6.8 | | 6.8 | 0.2 | 0.024 | 2.4 |

TABLE 3

| No. | | | | | | | | | | | |
|---|---|---|---|---|---|---|---|---|---|---|---|
| Age | | Number of measurements | | | | | | | | | |
| Sex | Factors | 1 | 2 | 3 | 4 | 5 | 6 | x̅ | SD | SD/ x̅ | ac. |
| No. 47 | Ps | 157 | 155 | 154 | 168 | 168 | | 160.4 | 7.0 | 0.044 | 4.4 |
| 78 y | Pd | 89 | 86 | 84 | 90 | 87 | | 87.2 | 2.4 | 0.027 | 2.7 |
| F | In Ps/Pd | 0.57 | 0.59 | 0.61 | 0.62 | 0.66 | | 0.6 | 0.03 | 0.057 | 5.7 |
| | PWV′ | 13.4 | 12.3 | 12.3 | 12.3 | 11.8 | | 12.4 | 0.6 | 0.047 | 4.7 |
| | PWV′² | 178.78 | 150.50 | 150.50 | 150.50 | 138.82 | | 153.8 | 14.8 | 0.096 | 9.6 |
| | PWV′²/Pd | 2.0 | 1.8 | 1.8 | 1.7 | 1.6 | | 1.8 | 0.2 | 0.089 | 8.9 |
| | PWV ori | 12.7 | 11.8 | 12.0 | 11.6 | 11.3 | | 11.9 | 0.5 | 0.044 | 4.4 |
| | CAVI | 13.4 | 13.8 | 13.3 | 12.6 | 12.7 | | 13.2 | 0.5 | 0.038 | 3.8 |
| | PWV pcm | 13.2 | 12.5 | 12.7 | 12.8 | 12.7 | | 12.8 | 0.3 | 0.021 | 2.1 |
| No. 48 | Ps | 148 | 139 | 139 | 128 | 123 | | 135.4 | 9.9 | 0.073 | 7.3 |
| 68 y | Pd | 92 | 88 | 92 | 82 | 86 | | 88.0 | 4.2 | 0.048 | 4.8 |
| M | In Ps/Pd | 0.48 | 0.46 | 0.41 | 0.45 | 0.36 | | 0.4 | 0.05 | 0.108 | 10.8 |
| | PWV′ | 10.3 | 10.7 | 10.0 | 9.8 | 11.4 | | 10.4 | 0.6 | 0.058 | 5.8 |
| | PWV′² | 106.66 | 114.02 | 100.00 | 96.90 | 128.85 | | 109.3 | 12.8 | 0.117 | 11.7 |
| | PWV′²/Pd | 1.2 | 1.3 | 1.1 | 1.2 | 1.5 | | 1.2 | 0.2 | 0.129 | 12.9 |
| | PWV ori | 9.6 | 10.2 | 9.3 | 9.7 | 10.9 | | 10.0 | 0.6 | 0.063 | 6.3 |
| | CAVI | 9.2 | 9.5 | 8.9 | 9.0 | 8.7 | | 9.1 | 0.3 | 0.034 | 3.4 |
| | PWV pcm | 9.8 | 9.9 | 9.0 | 9.1 | 9.4 | | 9.4 | 0.4 | 0.043 | 4.3 |
| No. 49 | Ps | 193 | 214 | 207 | 180 | 167 | 165 | 187.7 | 20.5 | 0.109 | 10.9 |
| 74 y | Pd | 92 | 103 | 99 | 89 | 88 | 82 | 92.2 | 7.7 | 0.083 | 8.3 |
| M | In Ps/Pd | 0.74 | 0.73 | 0.74 | 0.70 | 0.64 | 0.70 | 0.7 | 0.04 | 0.053 | 5.3 |
| | PWV′ | 12.4 | 13.9 | 12.2 | 12.8 | 13.0 | 11.8 | 12.7 | 0.7 | 0.056 | 5.6 |
| | PWV′² | 154.98 | 192.20 | 148.84 | 164.92 | 168.45 | 140.30 | 161.6 | 18.2 | 0.113 | 11.3 |
| | PWV′²/Pd | 1.7 | 1.9 | 1.5 | 1.9 | 1.9 | 1.7 | 1.8 | 0.2 | 0.087 | 8.7 |
| | PWV ori | 11.6 | 12.2 | 11.0 | 12.2 | 12.4 | 11.7 | 11.8 | 0.5 | 0.044 | 4.4 |
| | CAVI | 15.5 | 15.6 | 14.9 | 15.0 | 14.2 | 14.5 | 15.0 | 0.5 | 0.037 | 3.7 |
| | PWV pcm | 13.9 | 14.8 | 13.6 | 13.9 | 13.5 | 13.0 | 13.8 | 0.6 | 0.041 | 4.1 |
| No. 50 | Ps | 113 | 118 | 118 | 112 | 124 | | 117.0 | 4.8 | 0.041 | 4.1 |
| 62 y Pd | 79 | 80 | 81 | 75 | 83 | | 79.6 | 3.0 | 0.037 | 3.7 | |
| F | In Ps/Pd | 0.36 | 0.39 | 0.38 | 0.40 | 0.40 | | 0.4 | 0.02 | 0.048 | 4.8 |
| | PWV′ | 8.4 | 8.1 | 9.0 | 8.5 | 8.3 | | 8.5 | 0.3 | 0.039 | 3.9 |
| | PWV′² | 70.20 | 65.68 | 80.74 | 72.13 | 69.26 | | 71.6 | 5.6 | 0.078 | 7.8 |
| | PWV′²/Pd | 0.9 | 0.8 | 1.0 | 1.0 | 0.8 | | 0.9 | 0.1 | 0.086 | 8.6 |
| | PWV ori | 8.4 | 8.1 | 8.9 | 8.8 | 8.2 | | 8.5 | 0.4 | 0.043 | 4.3 |
| | CAVI | 7.4 | 7.3 | 7.6 | 7.6 | 7.6 | | 7.5 | 0.1 | 0.019 | 1.9 |
| | PWV pcm | 7.4 | 7.5 | 7.9 | 7.8 | 7.7 | | 7.7 | 0.2 | 0.029 | 2.9 |

The pulse wave data such as PWV′ and the blood pressure data such as Ps or Pd used by the arterial stiffness index calculating means 6 may not be a pulse wave propagation velocity or a blood pressure value itself but be waveform data of a pulse wave. Additionally, the existing pulse wave propagation velocity measured by the conventional method which has stored records and uses various pulse wave propagation velocities can be used.

The PWVpcm1 or the PWVpcm2 which is a calculated arterial stiffness index can be output, with past data or the like, as an index representing arterial stiffness to the display or the printer to be used for arterial stiffness diagnosis.

The description of the present invention should in no way construed to be limitative. Advantages, features, and uses of the invention will become apparent from the following description provided in connection with the drawings. Proper modifications without departing from a gist of the present invention are all included within the scope of the invention. It should be understood that various embodiments of the invention which have been described are only exemplary and thus not limitative in any way. The scope of the present invention should not be limited to the exemplary embodiments described above.

1. An arterial stiffness apparatus, comprising:
pulse wave detecting means;
pulse wave propagation velocity deciding means;
blood pressure detecting means; and
arterial stiffness index calculating means for calculating an arterial stiffness index to evaluate a degree of arterial stiffness based on a pulse wave propagation velocity and a blood pressure,
wherein the arterial stiffness index calculating means calculates a PWVpcm2 value by executing:
a first step of calculating a pulse wave propagation velocity PWVori after pressure calibration and CAVI=ln(Ps/Pd)×PWV′² based on highest blood pressure values Ps, lowest pressure values Pd, and pulse wave propagation velocities PWV′ before pressure calibration obtained from many test subjects, and deriving a regression equation where PWVori is represented by a quadratic equation of CAVI based on a relation between many PWVori values thus obtained and a CAVI value;
a second step of setting the regression equation obtained in the first step as an equation representing a arterial stiffness index PWVpcm1;
a third step of obtaining a PWVpcm1 value by substituting the CAVI value calculated in the first step into the equation representing the PWVpcm1 obtained in the second step, and deriving a regression equation where the PWVori is represented by a linear equation of the PWVpcm1 based on a relation between the PWVori value and the PWVpcm1 value;
a fourth step of setting the regression equation obtained in the third step as an equation representing an arterial stiffness index PWVpcm2; and
a fifth step of substituting the highest blood pressure value Ps, the lowest blood pressure value Pd, and the pulse wave propagation velocity PWV′ obtained by the pulse wave detecting means, the pulse wave propagation velocity deciding means, and the blood pressure detecting means, into the equation representing the PWVpcm2 obtained in the fourth step; and
the degree of arterial stiffness is evaluated based on the PWVpcm2.

2. An arterial stiffness evaluation apparatus according to claim 1, wherein:
the arterial stiffness index calculating means calculates the PWVpcm1 value by executing, in place of the third to fifth steps, a sixth step of obtaining a PWVpcm1 value by substituting the highest blood pressure value Ps, the lowest blood pressure value Pd, and the pulse wave propagation velocity PWV′ before the pressure calibration, obtained from the pulse wave detecting means, the pulse wave propagation velocity deciding means, and the blood pressure detecting means into the equation representing the PWVpcm1; and
the degree of arterial stiffness is evaluated based on the PWVpcm1 value.

3. An arterial stiffness evaluation apparatus according to claim 1 or 2, wherein the PWVori in the first step is obtained from the pulse wave propagation velocity PWV′ detected from carotid and tinea pulse waves.

4. An arterial stiffness evaluation apparatus according to claims 1 or 2, wherein the PWVori in the first step is obtained by pressure calibration with the lowest blood pressure value Pd set as 80 mmHg.

5. An arterial stiffness evaluation apparatus according to claims 1 or 2, wherein the pulse wave detecting means further comprises brachial pulse wave detecting means for detecting a pulse wave in the upper arm of an organism, and a popliteal pulse wave detecting means for detecting a pulse wave of poples of an organism.

6. An arterial stiffness evaluation apparatus according to claims 1 or 2, wherein a pulse wave detector equipped with a distortion sensor is used as the blood pressure detecting means.

7. An arterial stiffness evaluation apparatus according to claim 6, wherein the highest blood pressure is set as a blood pressure at a point of time when a negative notch unobserved in previous pulse waves is detected for the first time, and the lowest blood pressure is set as a blood pressure at a point of time when the notch disappears.

8. An arterial stiffness evaluation apparatus, comprising:
pulse wave detecting means;
pulse wave propagation velocity deciding means;
blood pressure detecting means; and
arterial stiffness index calculating means for calculating an arterial stiffness index to evaluate a degree of arterial stiffness based on a pulse wave propagation velocity and a blood pressure,
wherein the arterial stiffness index calculating means calculates at least one of an arterial stiffness index PWVpcm2 and an arterial stiffness index PWVpcm1 based on a highest blood pressure value Ps, a lowest blood pressure value Pd, and a pulse wave propagation velocity PWV′.

9. An arterial stiffness index calculation program for causing a computer to execute a process of calculating an arterial stiffness index which serves as an arterial stiffness index of an organism and a process of outputting the calculated arterial stiffness index to display means, by executing:
a first step of calculating a pulse wave propagation velocity PWVori after pressure calibration and CAVI=ln(Ps/Pd)×PWV′² based on a highest blood pressure value Ps, a lowest pressure value Pd, and a pulse wave propagation velocity PWV′ before pressure calibration obtained, and deriving a regression equation where PWVori is represented by a quadratic equation of CAVI from a relation between many obtained PWVori values and a CAVI value;
a second step of setting the regression equation obtained in the first step as an equation representing a arterial stiffness index PWVpcm1;
a third step of obtaining a PMVpcm1 value by substituting the CAVI value calculated in the first step into the equation representing the PWVpcm1 obtained in the second step, and deriving a regression equation where the PWVori is represented by a linear equation of the PWVpcm1 based on a relation between the PWVori value and the PWVpcm1 value;
a fourth step of setting the regression equation obtained in the third step as an equation representing an arterial stiffness index PWVpcm2; and
a fifth step of substituting the highest blood pressure value Ps, the lowest blood pressure value Pd, and the pulse wave propagation velocity PWV′ obtained by the pulse wave detecting means, the pulse wave propagation velocity deciding means, and the blood pressure detecting means, into the equation representing the PWVpcm2 obtained in the fourth step.

10. An arterial stiffness index calculation program for causing a computer to execute a process of calculating an arterial stiffness index based on a highest blood value Ps, a lowest blood pressure value Pd, and a pulse wave propagation velocity PWV′, based on at least one of an equation representing an arterial stiffness index PWVpcm1 and an arterial stiffness index PWVpcm2, and a process of outputting the calculated arterial stiffness index to display means.
